(19) United States
(12) Patent Application Publication
(10) Pub. No.: US 2025/0101354 A1
(43) Pub. Date: Mar. 27, 2025

(54) BIOREACTOR FOR PRODUCING A BIOLOGICAL MEDICAMENT, AND SUPPORT FOR SUCH A BIOREACTOR

(71) Applicant: CELLQUEST, Marchaux-Chaudefontaine (FR)

(72) Inventor: Guillaume Wallart, Mesandans (FR)

(21) Appl. No.: 18/729,983

(22) PCT Filed: Jan. 16, 2023
(86) PCT No.: PCT/EP2023/050898

§ 371 Date: Jul. 18, 2024

(30) Foreign Application Priority Data
Feb. 3, 2022 (FR) FR2200972

(51) Int. Cl. C12M 1/00; C12M 3/00

(52) U.S. Cl. CPC C12M 23/26; C12M 23/34; C12M 23/40; C12M 23/48; C12M 29/04

(57) ABSTRACT

A bioreactor for producing a biological medicament from a biological liquid that originates from a sample taken from a patient or a donor includes two flexible films joined locally via weld zones on the inner surfaces of the two films to form leak-tight barriers that mutually delimit a plurality of circulation channels connecting a plurality of compartments. At least one of the films comprises a plurality of interconnection vias, each of which opens into one of the channels or compartments. A support is used to support such a bioreactor.

### CROSS-REFERENCE TO RELATED APPLICATIONS

This application is a national phase entry under 35 U.S.C. § 371 of International Patent Application PCT/EP2023/050898, filed Jan. 16, 2023, designating the United States of America and published as International Patent Publication WO 2023/147988 A1 on Aug. 10, 2023, which claims the benefit under Article 8 of the Patent Cooperation Treaty of French Patent Application Serial No. FR2200972, filed Feb. 3, 2022.

### TECHNICAL FIELD

The present disclosure relates to the field of preparation of gene therapy medicaments and more precisely to the field of adoptive cellular immunotherapy based on genetic modification of a patient's T lymphocytes, immune cells, T lymphocytes, NK lymphocytes, macrophages, etc. or those of a donor so that they are able to recognize and destroy cancer cells.

### BACKGROUND

For such therapies, a patient's blood sample is used to isolate cells that play a major role in the immune system, controlling the body's defense system by identifying and destroying cells recognized as foreign to the organism, be they bacteria, viruses or cancer cells. First, blood is drawn from a patient or donor at a hospital or blood transfusion center. The cells of interest are harvested by a process called leukapheresis, or from a whole blood sample, in which white blood cells are isolated from other blood components. After quality control, these lymphocyte cells are sent to a specialized laboratory for genetic modification.

These isolated cells of interest are then genetically modified to express a specific chimeric protein (Chimeric Antigen Receptor, or CAR) on their surface. This enables them to both recognize cancer cells and activate themselves to destroy those same cancer cells. Once modified, the cells of interest are reinjected into the patient.

To carry out this modification, a new gene is introduced into the genome of the cells of interest, leading these cells to produce the desired chimeric protein. These cells are then cultured for cell multiplication.

The customized medicament prepared in this way is then administered to the patient in a single infusion.

The production of these modified cells in specialized laboratories requires the delicate operations of harvesting the cells of interest, isolating the cells, culturing them in a culture dish, periodically observing the health of the cells, suctioning, replacing and analyzing the culture medium, and so on.

U.S. Patent Application Publication No. US2017051238 describes a rigid culture vessel formed by molding a resin to form a three-dimensional structure with peripheral side faces pierced by connection ports. Transfer between two chambers 11, 12 is achieved by tilting the container to ensure flow through an external conduit 112 passing through connection ports provided on the side face of the container (paragraph [0081]), or by a difference in port height (paragraph [0082]). The container proposed in this application is therefore necessarily a thick form factor, with peripheral faces having a height greater than the cross-section of the connection ports.

U.S. Patent Application Publication No. US20200231918 describes a cartridge comprising a rigid PMMA lid (paragraph [0068]) defining a single cell culture chamber ranging in height from 0.5 mm to 100 mm, with pillars to support the top surface, forming spacers and rigid acrylate side surfaces.

U.S. Patent Application Publication No. US2014315303 describes a device for treating a biological sample. The device comprises a first sheet of material, a second sheet of material bonded to the first sheet of material, and a plurality of chambers defined between the first sheet of material and the second sheet of material. The chambers comprise a sample dissociation chamber comprising an inlet and an outlet; a waste collection chamber comprising an inlet in fluid communication with the outlet of the sample dissociation chamber, and a cell refining chamber comprising an inlet in fluid communication with the sample dissociation chamber and an outlet.

As with Publication No. US2017051238, the fluidic connection is made via peripheral fittings, through the peripheral edge of the device.

The prior art solutions have a three-dimensional configuration, obtained in particular by molding, and feature a thick edge traversed by orifices wherein supply or suction ducts are connected.

The construction of such devices presents difficulties in ensuring the perfect tightness of conduits passing through the edge to prevent leaks. In addition, the products contained in the chamber may remain in the peripheral edges, making it difficult to transfer the entire contents of one chamber to the next.

### BRIEF SUMMARY

In order to overcome these disadvantages, the present disclosure relates in its most general sense to a bioreactor for the production of a biological medicament from a biological fluid derived from a patient or donor sample.

It includes two flexible films joined locally by weld zones on the inner surfaces of the two films to form leak-tight barriers that mutually delimit a plurality of circulation channels connecting a plurality of compartments. At least one of the films comprises a plurality of interconnection vias each opening into one of the channels or compartments.

In one variant, at least one of the films is made of polyolefin.

In another variant, at least one of the films is made of polyethylene.

Preferably, at least one of the films is made of ultraviolet-transparent fluorinated ethylene propylene.

Advantageously, at least one of the films has an embossing that locally widens the distance between the films, in a duct zone delimited by two welds.

In one variant, the bioreactor also comprises at least one filtration zone formed by a filtering piece interposed between the upper film and the lower film and welded over at least part of its periphery to at least one of the films.

Advantageously, the filter part is welded to one of the films over part of its periphery, the non-welded part opening onto a zone surrounded by weld lines locally sealing the upper film and the lower film to form a feed zone for the filter, the film opposite that to which the filter is welded also having a zone surrounded by weld lines locally sealing the upper film and the lower film to form an outlet zone for the filter.

In one variant, the vias are extended by a tubular injection or suction pin.

According to another variant, one zone of at least one of the films is surrounded by weld lines and has a functionalized inner surface.

The disclosure also relates to a support for a bioreactor including a rigid frame having means for attaching a bioreactor and at least one element having fluidic connectors, the element being movable between a separated position and a position in which the fluidic connectors are joined with the vias of the bioreactor.

Advantageously, the frame has windows corresponding to the positions of the compartments, for the passage of an actuation means acting on the film surface of the bioreactor.

In one variant, the support comprises at least one sensor arranged in a zone delimited by welds, the sensor being extended by wires opening onto the edge of the bioreactor.

In another variant, the support comprises a unique identifier that can be read optically or by radio-frequency communication.

### BRIEF DESCRIPTION OF THE DRAWINGS

The present disclosure will be better understood on reading the following description, which concerns a non-limiting exemplary embodiment that is shown by the appended drawings, in which:

FIG. 1 shows a top view of a bioreactor according to a first embodiment of the disclosure;

FIG. 2 shows a top view of a bioreactor support in an open position according to a first embodiment of the disclosure;

FIG. 3 shows a top view of a bioreactor support in a closed position according to the first example embodiment of the disclosure;

FIG. 4 shows a top view of a bioreactor positioned in a bioreactor support;

FIG. 5 shows an enlarged perspective view of the fluidic connection between the support and the bioreactor;

FIG. 6 shows an enlarged view of forming a channel;

FIG. 7 shows a bottom view of the upper film during a first step of forming a filter zone;

FIG. 8 shows a top view of the bioreactor during a second step of forming a filter zone;

FIG. 9 shows a top view of the bioreactor during a third step of forming a filter zone;

FIG. 10 shows a top view of the bioreactor during a fourth step of forming a filter zone; and

FIG. 11 shows a top view of a bioreactor according to a second example embodiment of the disclosure.

### DETAILED DESCRIPTION

The present disclosure concerns a bioreactor designed to perform a succession of physical, chemical or biochemical treatments for the production of Car-T, Car-NK, Car-M, bio-identical proteins, antibodies, stem cells and, more generally, sequences of treatments of a biological fluid obtained from a patient or donor for the preparation of a gene therapy medicament.

The disclosure is based on the principle of forming a flexible pouch, formed from two flexible films having peripheral weld lines and zones to form a pouch closed along its entire peripheral edge, as well as local weld lines and zones where the two films are joined to form a leak-tight barrier separating other zones, where the two films can be locally spread to form compartments or channels allowing fluid circulation. The flexibility of the films makes it possible to exert an external action to expel content from one compartment to another compartment or to an outlet duct by exerting pressure on the film at the relevant zone, and also makes it possible to open or close circulation channels by exerting pressure on a transverse line of a channel. Some film surfaces can be functionalized to act chemically or biochemically on the fluid circulating in the bioreactor. The multi-compartment flexible pouch design, which defines a circulation circuit by means of simple local welds, makes for a highly economical bioreactor that can be used for both sample storage and automatic processing in an automated machine, thereby considerably reducing the cost of producing personalized medicaments.

### Description of an Example of a Bioreactor According to a First Variant

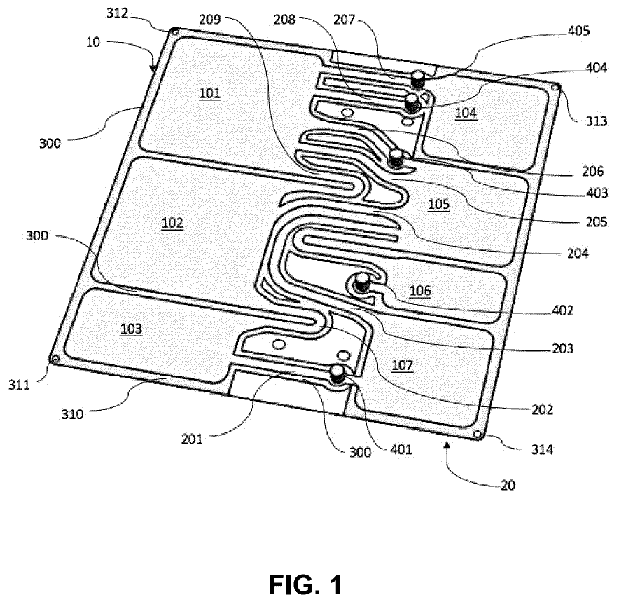

FIG. 1 shows a top view of a first example of a bioreactor, showing the compartments (101 to 107) and channels (201 to 209) through a transparent view.

This bioreactor, designed for the preparation of biological medicaments in a closed system, is formed by a pouch made up of two main films (10, 20), hereinafter conventionally referred to as “upper film (10) and lower film (20),” although the pouch is perfectly reversible. These two films (10, 20) are welded thermally or by ultrasonic (US) or high-frequency (HF) welding along lines or larger zones (300), to divide the pouch into compartments (101 to 107) linked by channels (201 to 209). The term “welded” includes gluing or any other assembly technique that locally joins the lower surface of the upper film (10) and the upper surface of the lower film (20) to form a local leak-tight barrier. The periphery of the pouch is welded to form a sealed edge whose thickness corresponds to the thickness of the two films (10, 20). Optionally, the pocket can be made from a single film folded over to form two superimposed panels (10, 20) connected by a common edge.

When the pouch is empty, its thickness corresponds to the cumulative thickness of the films that make it up, which are arranged in parallel planes with no peripheral side faces.

Each compartment (101 to 107) can be isolated from the others by pressing on these channels (201 to 209), for example, by a system described later.

Similarly, the contents of one compartment (101 to 107) can be transferred into a compartment (101 to 107) to which it is connected by a channel (201 to 209) by removing the pressing piece that closes the channel and applying pressure to this compartment (201 to 209). For example, in the example shown in FIG. 1, by removing the pressure from the channel (201) and pressing on the compartment (107), liquid can be transferred from the compartment (107) to the compartment (103), or, by maintaining the pressure on the channel (201) and releasing the pressure on the channel (203), liquid can be transferred from the compartment (107) to the compartment (102).

The bioreactor also features injection sites (401 to 405) distributed over the transfer channels (201, 207, 208), enabling the compartments to be filled with different liquids. These injection sites (401 to 405) are formed by vias passing through one of the films (10, 20) perpendicularly to its surface, into which are inserted tubular pins constituting a fluidic connector arranged on one or two of the faces of the pouch, to extend perpendicularly to the median plane of the pouch. These injection sites (401 to 405) correspond, for example, to transfusion bag standards.

The bioreactor also features a welded peripheral strip (310), the corners (311, 312, 313, 314) of which are pierced by holes for cooperating with lugs on a support to hold the bioreactor on a dedicated support.

The materials of both films are suitable for use.

For use in producing cell culture factors or CarT Cell or Car-NK Cell, the upper film (10) is made of polyolefin and the lower film (20) of polyethylene, both medical-grade.

These two materials are chosen for their permeability to CO₂ and O₂ (for cell culture) and to UV-B to induce apoptosis if required.

The lower polyethylene film (20) enables cell proliferation or adhesion to be controlled, thanks to the material's optically transparent quality.

In the case of FEP film, which is transparent to UV-C, it is possible to induce cell necrosis or to sterilize the contents by applying ultra-violet radiation.

### Description of the Bioreactor Support

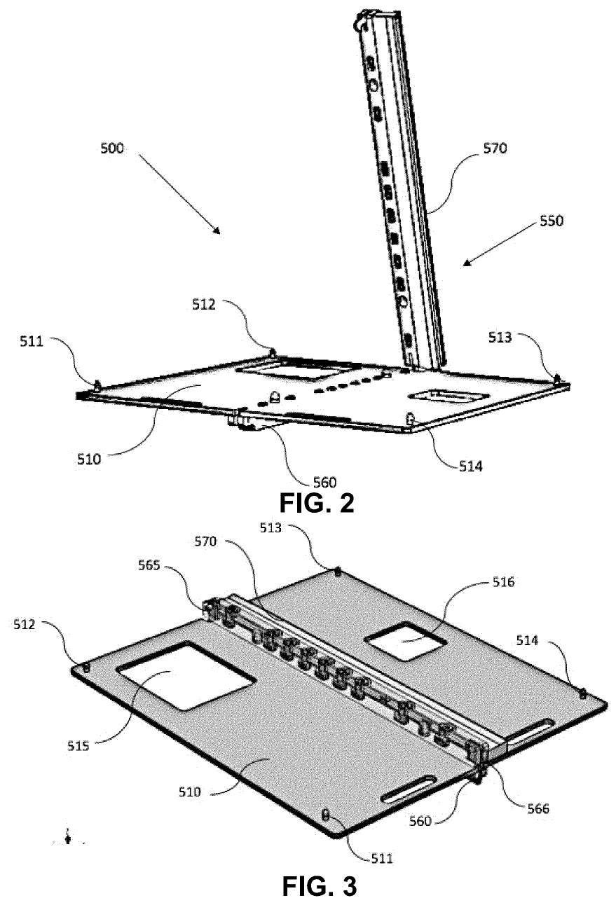

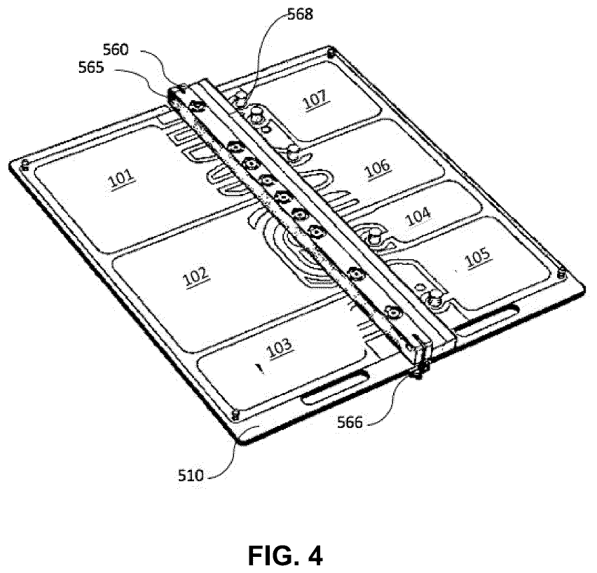

FIGS. 2 to 4 illustrate the configuration of the support, in the open and closed positions without a bioreactor therein, and closed with a bioreactor therein, respectively.

This support (500) consists of a plastic or metal tray (510), approximately 5 mm thick, and an articulated clamp (550) with a fixed arm (560) integral with the tray (510) and a pivoting arm (570).

The top surface of the tray (510) features four peripheral pins (511 to 514) for positioning the bioreactor bag on a support (500) via the four peripheral holes (311 to 314).

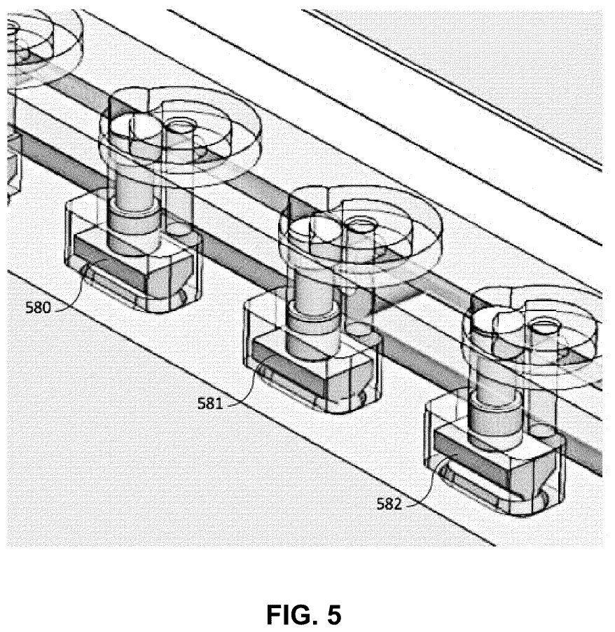

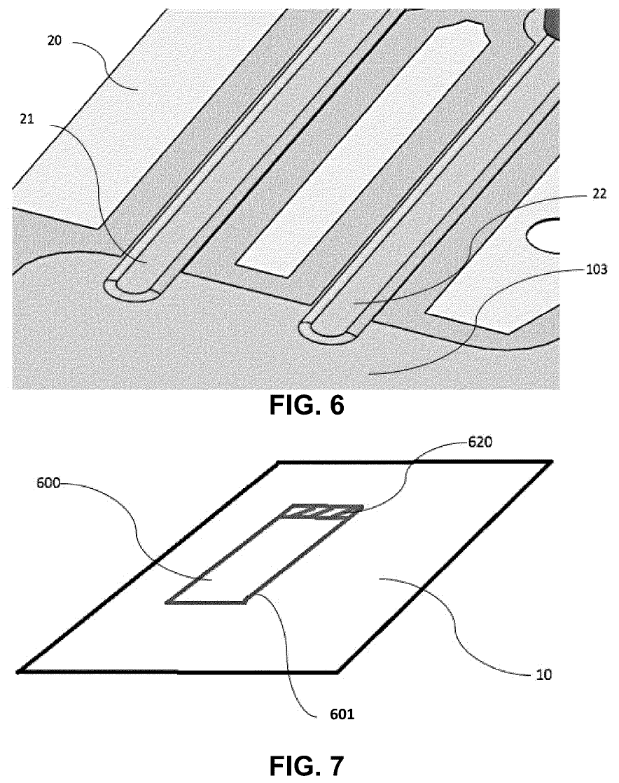

The fixed arm (560) is formed by an aluminum bar that allows the mounting of silicone pins at injection sites (401 to 405) located below the bioreactor transfer channels. The pivoting arm (570) is formed by a second aluminum bar, which is integral with the fixed arm (560) via a joint (565). In the closed position, the pivoting arm (570) locks to the front (566) of the fixed arm (560). In the top bar, a series of ¼-turn mechanisms press a metal anvil onto each silicone pin as shown in FIG. 5.

The tray (510) is opaque and has cut-outs (515, 516) positioned under the compartments (101 to 107) for interaction between external equipment and the lower surface of the bioreactor, for example, light or optical interaction (excitation in a given wavelength or visible or infrared spectrum, observation) or mechanical interaction (pressure, vibration, etc.). These cut-outs (515, 516) can also be designed to transmit vibration to a single compartment, in order to loosen adherent cells or mix the liquid contained in that compartment.

The actuator used for this operation may be a surface-mounted loudspeaker or a motor with eccentric, alternately excited electromagnet, or rotating cams.

Certain cut-outs (515, 516) on the support can be filled with a plate made of materials transparent to UV-C or UV-B (FEP, PE, glass, etc.), enabling irradiation by positioning a card equipped with UV-C or B LEDs under the support for sterilization, necrosis or apoptosis of the cells present in the corresponding compartment.

The compartments (101 to 103) are isolated from the other four compartments (104 to 107) by a gasket (568), enabling two thermostatically-controlled zones to be partitioned off at two different temperatures, for example, the left-hand zone at 37° C. and the right-hand zone at 4° C.

Once the bioreactor has been positioned on this support (500), the transfer channels from one compartment to another can be closed by pressurizing the anvil (580 to 581) on the silicone pin by an actuator present on the machines into which these supports will be threaded.

Embossings (21, 22) on the lower film (20), in line with the transfer channels (202), enable the liquid to pass quickly and easily from one compartment to another without applying high pressure to the pouches. The embossing is sufficient, but limited, to avoid folds forming when the sheets are crushed under the anvil. In the example, the channels are 8 mm wide and the embossing is 0.3 mm high.

### Filtration Zone

The design principle of the bioreactor according to the disclosure makes it easy to incorporate a filter zone. To achieve this, a filter element, such as a porous membrane (600), is inserted locally between the upper film (10) and the lower film (20). This filter membrane (600) is made, for example, of a rectangular piece with a mesh size of 1 to 4 pm for cell concentration.

This filter membrane (600) is positioned to separate along a transverse median plane a compartment (101 to 107), the upper volume of which, between the filter membrane (600) and the upper film (10), will communicate fluidically with a compartment or channel isolated from the lower volume, and the lower volume of which, between the filter membrane (600) and the lower film (20), will communicate with another compartment or channel, isolated from the first by a weld.

In the first step, the filter membrane (600) is placed against the lower surface of the upper film (10). The filter membrane (600) is thermally welded on one side (620) to a weld strip (601) approximately 5 mm wide.

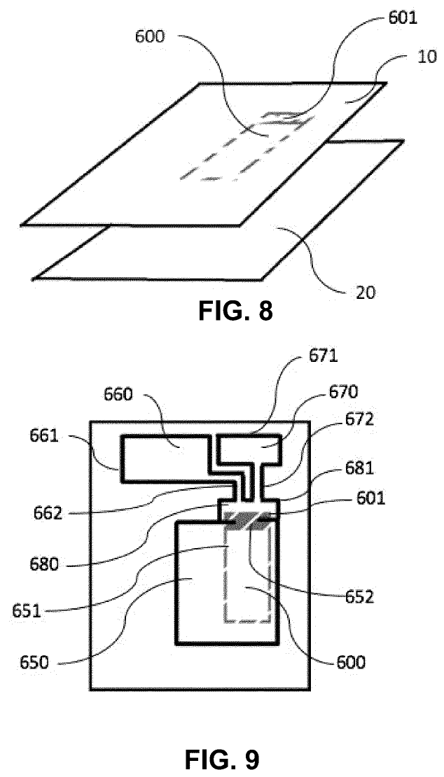

The second step, illustrated in FIG. 8, involves superimposing the lower film (20) and the upper film (10). The upper film (10) with its filter membrane (600) is turned upside-down (filter underneath) and positioned on the lower film (20).

The third step, illustrated in FIG. 9, involves creating local weld lines in a configuration formed by:
an open rectangular segment (651) delimiting a compartment (650) surrounding the filter membrane (600), and having an opening (652) at the weld strip (601) of the filter membrane (600);
a rectangular segment (681) delimiting a second compartment (680) communicating in the lower part of the filter between the filter membrane (600) and the lower film (20) with the compartment (650) via the opening (652);
a rectangular segment (661) delimiting a third compartment (660) communicating with the second compartment (680) via a channel (662); and
a rectangular segment (671) delimiting a fourth compartment (670) communicating with the second compartment (680) via a channel (672).

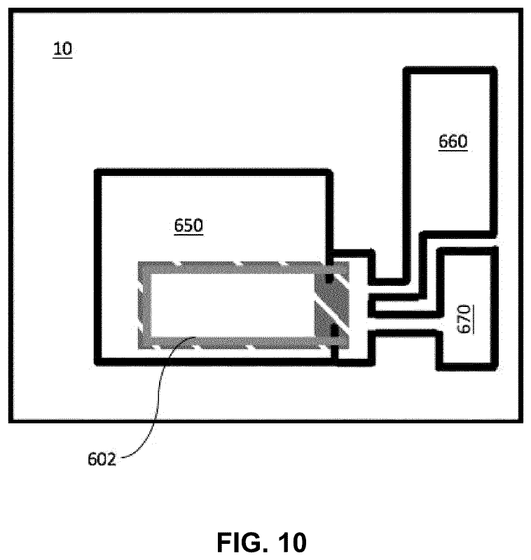

The last step is illustrated in FIG. 10. A mold is then used to close the filter by welding the upper film (10) to the filter membrane (600) along a peripheral line (602) completing the weld strip (601).

The liquid present in the lower compartment (650) can only enter one of the compartments (660, 670) by passing through the filter membrane (600).

If cells are grown in compartment (650), and the channels (662) and (672) between the lower compartment (650) and the other compartments (660) and (670) respectively are clamped. Compartment (660) is empty and compartment (670) contains a different culture medium to that used in compartment (650).

A programmable logic controller into which the bioreactor is inserted performs a series of actions:
Step 1: The programmable logic controller controls the opening of the channel (662) between the culture compartment (650) and the empty compartment (660).
Step 2: The surface of the compartment (650) is pressed against the culture to transfer the contents of the compartment (650) to the compartment (660). Passage is via the filter, with the filter membrane (600) interposed between the lower part of the compartment (650) and the channel (662). If the filter membrane (600) is sized to retain the cells (e.g., 0.65 pm filter), the cells remain in the compartment (650), while the medium is transferred to the compartment (660).
Step 3: The passage between compartments (650) and (660) is closed by clamping the channel (662), and the channel (672) between the compartment (650) and the compartment (670) is opened. The medium contained in the compartment (670) is transferred to re-suspend the cells contained in the compartment (650), which had previously been emptied of its culture medium.

Steps 1 and 2 are used to concentrate cells (before transduction, for example). Steps 1, 2 and 3 are used to change the medium.

### Bioreactor Variant

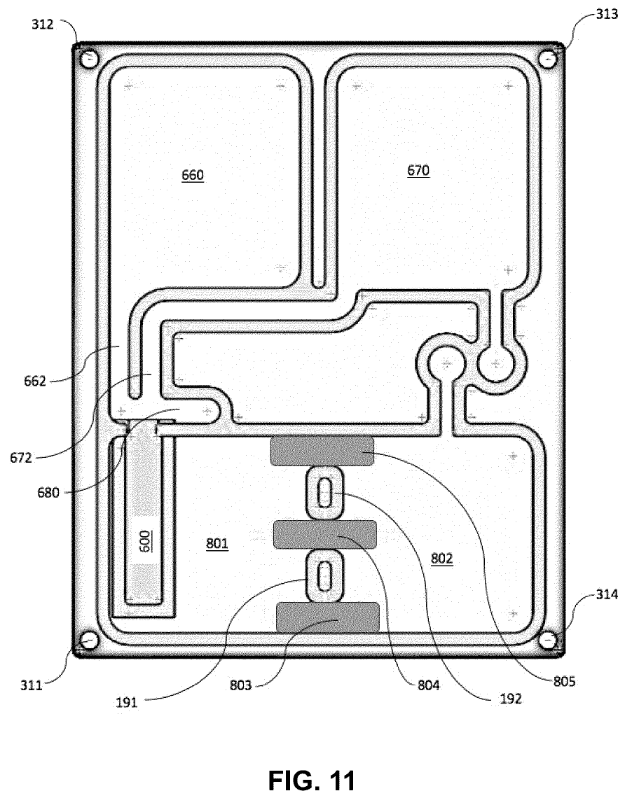

FIG. 11 shows a variant of the bioreactor, whose implementation and technical features in common with the previous variant are not detailed further.

The lower part of this bioreactor shows a pocket containing the filter membrane (600) described above.

This pocket also contains two oblong welds (191, 192) that divide the compartment into two zones (801, 802) connected by three channels (803 to 805).

Hot-embossed bosses form connection channels (803 to 805) measuring a few tenths of a mm between these zones.

Successive presses between the right and left zones allow transfer from left to right and then right to left to mix the liquid inside.

1. A bioreactor for production of a biological medicament from a biological liquid that originates from a sample taken from a patient or donor, the bioreactor comprising a multi-compartment flexible pouch formed by two flexible films joined locally by weld zones on inner surfaces of the two flexible films to form leak-tight barriers that mutually delimit a plurality of circulation channels connecting a plurality of compartments, at least one of the two flexible films comprising a plurality of interconnection vias each opening into one of the plurality of circulation channels or the plurality of compartments, the plurality of interconnection vias passing through one of the two flexible films perpendicularly to a surface thereof, into which are inserted tubular pins constituting a fluidic connector arranged on one or two of the faces of the multi-compartment flexible pouch, to extend perpendicularly to a median plane of the multi-compartment flexible pouch.

2. The bioreactor of claim 1, wherein at least one of the two flexible films is made of polyolefin.

3. The bioreactor for the production of a biological medicament of claim 1, wherein at least one of the two flexible films is made of polyethylene.

4. The bioreactor of claim 1, wherein at least one of the two flexible films is made of ultraviolet-transparent fluorinated ethylene propylene.

5. The bioreactor of claim 1, wherein at least one of the two flexible films has an embossing locally widening a distance between the two flexible films in a duct zone delimited by two welds.

6. The bioreactor of claim 1, further comprising at least one filtration zone formed by a filtering piece interposed between the two flexible films and welded over at least part of a periphery of the filtering piece to at least one of the two flexible films.

7. The bioreactor of claim 6, wherein the filtering piece is welded to one of the two flexible films over part of its periphery, a non-welded part opening onto a zone surrounded by weld lines locally sealing the two flexible films to form a feed zone for the filtering piece, a flexible film opposite that to which the filtering piece is welded also having a zone surrounded by weld lines locally sealing the two flexible films to form an outlet zone for the filtering piece.

8. The bioreactor of claim 1, wherein the plurality of interconnection vias are extended by an injection tube or a suction pin.

9. The bioreactor of claim 1, wherein a zone of at least one of the two flexible films is surrounded by weld lines and has a functionalized inner surface.

10. A bioreactor assembly, comprising:
a bioreactor according to claim 1; and
a support, including a rigid frame having a mechanism for attaching a bioreactor and at least one element having fluidic connectors, the at least one element being movable between a separated position and a position in which the fluidic connectors are joined with the plurality of interconnection vias of the bioreactor.

11. The assembly of claim 10, wherein the rigid frame has windows corresponding to positions of compartments of the plurality of compartments, for passage of an actuator for acting on a surface of the bioreactor.

12. The assembly of claim 10, further comprising at least one sensor arranged in a zone delimited by welds, the at least one sensor being extended by wires opening onto an edge of the bioreactor.

13. The assembly of claim 10, further comprising a unique identifier that can be read optically or by radio-frequency communication.
